(19) United States
(12) Patent Application Publication
(10) Pub. No.: US 2006/0067958 A1
(43) Pub. Date: Mar. 30, 2006

(54) Pharmaceutical topical gel compositions

(75) Inventors: Donna L. Valencia, Pasig City (PH); Sarah A. Calapini, Mandaluyong City (PH); Kennie U. Dee, Quezon City (PH)

Correspondence Address: AQUILLA PATENTS & MARKS, PLLC, 221 COE HILL ROAD, CENTER HARBOR, NH, 03326-3605, US

(73) Assignee: Unilab PharmaTech, Ltd., Central Hong Kong (CN)

(21) Appl. No.: 10/949,591

(22) Filed: Sep. 24, 2004

(51) Int. Cl. A61K 9/14

(52) U.S. Cl. 424/401; 424/488

(57) ABSTRACT

Topical alcoholic gel compositions containing dissolved pharmaceutical actives, which, in their preferred form, have excellent clarity. The alcoholic gel contains less than 20% w/w water, and uses hydroxypropylcellulose and non-neutralized, partially neutralized, or fully neutralized acrylic acid-based polymer as effective synergistic gelling system, and diols and/or triols as an optional tertiary synergistic gelling agent.

### BACKGROUND OF THE INVENTION

1. Field of the Invention

The present invention relates to new and useful topical compositions containing dissolved pharmaceutical actives in a dermatologically acceptable alcoholic gel base. Clear compositions can be made and are preferred. Hereafter, we will use alcoholic gel to refer to a composition with less than 20% w/w water, and hydroalcoholic gel to a composition with more than 25% w/w water.

2. Description of Related Art

Alcohol, particularly ethanol, is a known permeation enhancer for topical drugs. Increased rate of drug absorption leads to faster onset of action and enhanced efficacy. Ethanol is currently used in gel formulations, but generally as a hydroalcoholic gel with more than 30% w/w water. There is a need for an alcoholic gel containing dissolved drugs with very low level of water, preferably less than 20% w/w, for various reasons. First, the presence of high level of water in the composition can retard the absorption rate. Second, the drug may not be soluble in the presence of water such as for example when the drug preferentially forms an insoluble hydrate. Third, some drugs degrade rapidly in the presence of water. For example, hydrocortisone degrades in water but has acceptable stability in alcohol (J. Pharm Sci, 1978, vol. 67, 299-302). Because of the incompatibility of hydrocortisone with water, commercial preparations are normally formulated as oil-in-water or water-in-oil emulsions where hydrocortisone is dissolved in the oil phase to reduce its interaction with water. There are also a number of drugs, such as the corticosteroids, which are converted to their ester prodrugs for use in topical formulations to improve and/or modulate their rate of absorption; these esters can hydrolyze in the presence of water especially when stored at elevated temperature.

The thickener most commonly used in hydroalcoholic gels is an acrylic acid-based polymer such as carbomer that requires neutralization with a base for thickening. However, as the amount of water in the formulation decreases, it becomes increasingly difficult to form a gel. In particular, it is necessary to significantly increase the amount of carbomer used. For example, more than three times more carbomer may be needed when the amount of water is below 10% w/w compared to a hydroalcoholic gel formulation with more than 30% w/w water. Also, the choice of base for neutralization of the carbomer becomes limited. At high alcohol levels, alkaline bases such as sodium hydroxide and most of the alkanolamines such as triethanolamine form a precipitate with the carbomer. The preferred bases for alcoholic gels are the amine bases diisopropanolamine, triisopropanolamine, and tetrahydroxypropylethylenediamine. The base/acrylic acid-based polymer neutralization weight ratios are 1.2 for diisopropanolamine, 1.5 for triisopropanolamine, and 2.3 for tetrahydroxypropylethylenediamine.

A higher level of carbomer in an alcoholic gel requires a higher level of base for neutralization which, compounded by the fact that alcohol is a penetration enhancer, can lead to skin irritation. It is therefore desirable to limit the amount of base in the formulation or to eliminate the base entirely. In the present invention, we have surprisingly found that hydroxypropylcellulose can be used synergistically with non-neutralized, partially neutralized, or fully neutralized acrylic acid-based polymer to thicken an alcoholic gel, with diols and/or triols as optional tertiary synergistic gelling agent. One embodiment of this instant invention is the use of non-neutralized or partially neutralized acrylic acid-base polymer with hydroxypropylcellulose, and optionally diols and/or triols, to form an alcoholic gel. A second embodiment is the use of low levels of fully neutralized acrylic acid-base polymer with hydroxypropylcellulose, and optionally diols and/or triols, to form an alcoholic gel.

While it is possible to prepare topical alcoholic compositions of dissolved drugs as liquid solutions instead of gels, liquid solutions are generally difficult to dispense and can lead to spillage and overdosing. Therefore, it is highly desirable to formulate alcoholic preparations as gels.

U.S. Pat. No. 4,393,076 describes ketoprofen gel compositions with excellent percutaneous absorption comprising: 2-40% of a glycol; 20-55% water; up to 60% of a lower alcohol; 0.5-5% gelling agent selected from carbomer, hydroxyethylcellulose, methylcellulose, carboxymethylcellulose, hydroxypropylcellulose, and alginic acid-propylene glycol ester; and 0.1-3% of a neutralizing agent. U.S. Pat. No. 4,393,076 does not teach that hydroxypropylcellulose can be used synergistically with non-neutralized, partially neutralized, and fully neutralized acrylic acid-based polymer to thicken an alcoholic gel with less than 20% w/w water, with diols and/or triols as optional tertiary synergistic gelling agent.

U.S. Pat. No. 4,678,666 discloses a piroxicam gel ointment with 30-60% w/w water, comprising: a lower alkanol; a polyhydric alcohol; carbomer; an alkanolamine to dissolve piroxicam and neutralize the carbomer; and a film-former selected from polyvinylpyrrolidone, hydroxyethylcellulose, carboxymethylcellulose. U.S. Pat. No. 4,678,666 does not teach that hydroxypropylcellulose can be used synergistically with non-neutralized, partially neutralized, or fully neutralized carbomer to thicken an alcoholic gel, with diols and/or triols as optional tertiary synergistic gelling agent. Further, as shown in Example 5 below, polyvinylpyrrolidone, hydroxyethylcellulose, and carboxymethylcellulose do not have any synergistic thickening effect when used with an acrylic acid-based polymer in alcoholic gels.

U.S. Pat. No. 5,436,241 discloses a piroxicam gel ointment comprising: 30-60% of C2-C4 alkanol; C2-C6 alkylene glycol as detackifier/humectant; carbomer; tetrahydroxypropylethylenediamine to dissolve piroxicam and neutralize the carbomer; and optionally hydroxypropylcellulose as an additional gelling agent. All the compositions described in U.S. Pat. No. 5,436,241 contain more than 25% w/w water, and sufficient tetrahydroxypropylethylenediamine to dissolve the piroxicam and neutralize the carbomer. Although hydroxypropylcellulose is mentioned as an additional thickening agent in U.S. Pat. No. 5,436,241, it is an optional ingredient and not an essential one as in the present invention. U.S. Pat. No. 5,436,241 does not teach that hydroxypropylcellulose can be used synergistically with non-neutralized, partially neutralized, and fully neutralized acrylic acid-based polymer to thicken an alcoholic gel with less than 20% w/w water comprising any dissolved drug other than piroxicam, with diols and/or triols as optional tertiary synergistic gelling agent.

U.S. Pat. No. 5,460,834 describes ophthalmic physiological tear compositions having high viscosity containing bicarbonate, at least one cellulosic polymer, and at least one carbomer. U.S. Pat. No. 5,460,834 discloses that fully neutralized carbomer can be used with cellulosic polymers to synergistically thicken aqueous solutions. The preferred cellulosic polymers are hydroxypropylmethylcellulose, hydroxyethylcellulose, methylcellulose, and hydroxypropylcellulose. All compositions described in U.S. Pat. No. 5,460,834 are non-alcoholic with greater than 95% w/w water. U.S. Pat. No. 5,460,834 therefore does not teach how to prepare alcoholic gels of the present invention. As shown in Example 5 below, only hydroxypropylcellulose is synergistic with acrylic acid-based polymers in alcoholic gels; hydroxypropylmethylcellulose, hydroxyethylcellulose, and methylcellulose show no synergism. Also, the present invention shows that hydroxypropylcellulose is synergistic even with non-neutralized or partially neutralized acrylic acid-based polymers, whereas all the compositions in U.S. Pat. No. 5,460,834 are with fully neutralized carbomer.

WO9939713 describes a piroxicam-containing hydroalcoholic gel composition with improved absorption comprising: 40-60% lower alkanol; 0.1-5% of hydroxypropylcellulose or hydrophobic derivatives of hydroxypropylmethylcellulose, optionally comprising hydroxypropylmethylcellulose or carbomer, as polymer vehicle; 0.1-20% of at least one absorption enhancer; 0.1-5% of at least one pH controlling agent; and water. WO9939713 does not teach how to prepare alcoholic gels of the present invention. First, water is an essential ingredient in WO9939713 and all compositions described in WO9939713 have 28-53% w/w water, in contrast with the present invention where water is less than 20% w/w. Second, hydroxypropylcellulose is an essential ingredient in WO9939713 but the carbomer is optional, in contrast with the present invention where both hydroxypropylcellulose and an acrylic acid-based polymer are essential ingredients.

### SUMMARY OF THE INVENTION

The present invention provides novel topical alcoholic gel compositions containing dissolved actives which, in their preferred form, have excellent clarity. The compositions of the instant invention contain less than 20% w/w water, and use hydroxypropylcellulose and non-neutralized, partially neutralized, or fully neutralized acrylic acid-based polymer as synergistic gelling agents, and diols and/or triols as an optional tertiary synergistic gelling agent. With the gelling system of the present invention, the amount of base used to neutralize the acrylic acid-based polymer can be reduced or eliminated altogether. In addition, if fully neutralized acrylic acid-based polymer is used, a lower level of this polymer is required when used with the gelling system of the present invention. Finally, the gelling system of the present invention allows one to achieve higher viscosity in an alcoholic gel than what is possible with the fully neutralized acrylic acid-based polymer approach of prior art.

### DETAILED DESCRIPTION OF THE INVENTION

The first essential component of the present invention is a dissolved pharmaceutical active ingredient suitable for topical administration. These actives are present at safe and effective levels from about 0.02% w/w to about 30% w/w. Such actives include, but are not limited to, analgesics, counter-irritants, anti-inflammatory agents, antipruritics, anesthetic agents, antifungals, antiviral agents, antibiotics, and mixtures thereof. Specific drugs useful in the composition of the present invention include, but are not limited to, methyl salicylate, menthol, camphor, capsaicin and its derivatives, diphenhydramine and its salts, ibuprofen, ketoprofen, diclofenac, benzocaine, lidocaine, lidocaine hydrochloride, prilocaine, prilocaine hydrochloride, hydrocortisone, hydrocortisone aceponate, hydrocortisone acetate, hydrocortisone butyrate, hydrocortisone valerate, betamethasone dipropionate, betamethasone valerate, clobetasol propionate, flucinolone acetonide, triamcinolone acetonide, and halobetasol propionate.

The composition contains as an essential component a C2-C3 alkanol. Ethanol is preferred. The alkanol is present in an amount greater than 40% w/w, preferably greater than 50% w/w. The alcohol serves to dissolve the active ingredients and enhance skin permeation.

The gelling system of the present invention comprises hydroxypropylcellulose and an acrylic acid-based polymer. The acrylic acid-based polymers are high molecular weight, crosslinked homopolymers and copolymers of acrylic acid. The homopolymers are also known as carbomers and are available from B.F. Goodrich under the trade name Carbopol 940, Carbopol 980, Carbopol 934, Carbopol 941, Carbopol 981, and Ultrez 10. The acrylic acid-based copolymers are copolymer of acrylic acid with alkyl methacrylate, also available from B.F. Goodrich as ETD 2020, and the Pemulen series. Preferably, the acrylic acid-based polymer and hydroxypropylcellulose are both present individually at a concentration between 0.2% w/w and 2% w/w, most preferably between 0.3% w/w and 1.3% w/w. The preferred ratio of hydroxypropylcellulose to acrylic acid-based polymer is 0.5 to 4, most preferably 0.7 to 3. The viscosity of the final composition at a shear rate of 2.5/sec is at least 3,000 cps, preferably greater than 8,000 cps, and most preferably greater than 12,000 cps.

In addition to hydroxypropylcellulose and an acrylic acid-based polymer, the alcoholic gels of the present invention can optionally contain a third synergistic gelling agent selected from C2-C6 alkylene glycol or diols, C3-C6 alkylene triols, and mixtures thereof. Preferred diols include propylene glycol, butylene glycol, hexylene glycol, and mixtures thereof. The preferred triol is glycerin. In general, the third gelling agent when used is present in an amount from about 5% w/w to about 35% w/w; most preferably, from about 10% w/w to about 30% w/w.

Water is not an essential ingredient, but may be present in small amount to faciliate dispersion of the acrylic acid-based polymer and hydroxypropylcellulose. The water is present in an amount less than 20% w/w, preferably less than 15% w/w, and most preferably less than 10% w/w.

The composition may optionally contain an amine base selected from, but not limited to, tetrahydroxypropylethylenediamine, diisopropanolamine, triisopropanolamine, and mixtures thereof. The composition may further contain penetration enhancers, solubilizers, preservatives, emollients, moisturizers, humectants, antioxidants, dyes, and fragrances.

### EXAMPLE 1

| | 1-A | 1-B | 1-C | 1-D | 1-E | 1-F | 1-G |
|---|---|---|---|---|---|---|---|
| Ingredients | % w/w | % w/w | % w/w | % w/w | % w/w | % w/w | % w/w |
| ETD 2020 | 1 | — | — | 1 | 1 | — | 1 |
| Hydroxypropylcellulose | — | 0.75 | — | 0.75 | — | 0.75 | 0.75 |
| Propylene Glycol | — | — | 20 | — | 20 | 20 | 20 |
| Water | 5 | 5 | 5 | 5 | 5 | 5 | 5 |
| Ethanol | q.s. | q.s. | q.s. | q.s. | q.s. | q.s. | q.s. |
| Viscosity (2.5/sec) | 51 | 510 | 25 | 4,740 | 51 | 587 | 8,040 |
| cps | | | | | | | |

The formulations were prepared as follows: hydroxypropylcellulose and/or ETD 2020 when present in the formulation were added to the ethanol-water mixture with stirring. When the polymer(s) was fully hydrated, propylene glycol if present in the formulation was added and mixing continued until the mixture was homogeneous. For Example 1-C, which does not contain a polymer, the ethanol, water, and propylene glycol were combined directly and mixed. Viscosity was measured using a Haake VT550 viscometer at a shear rate of 2.5/sec.

Comparing Example 1-D with Examples 1-A and 1-B clearly shows the synergy of hydroxypropylcellulose and the acrylic acid-based polymer ETD 2020. Examples 1-E, 1-C, and 1-A show that propylene glycol does not exhibit synergism with the acrylic acid-based polymer. Examples 1-F, 1-B, and 1-C demonstrate that propylene glycol when combined with hydroxypropylcellulose show no synergism. However, comparing Example 1-G with Example 1-D shows that propylene glycol is an effective tertiary gelling agent to the already synergistic hydroxypropylcellulose-acrylic acid-based polymer combination.

### EXAMPLE 2

| | 2-A | 2-B | 2-C | 2-D | 2-E | 2-F | 2-G |
|---|---|---|---|---|---|---|---|
| Ingredients | % w/w | % w/w | % w/w | % w/w | % w/w | % w/w | % w/w |
| Methyl salicylate | 10 | 10 | 10 | 10 | 10 | 10 | 10 |
| Menthol | 7 | 7 | 7 | 7 | 7 | 7 | 7 |
| ETD 2020 | 1 | — | — | 1 | 1 | — | 1 |
| Hydroxypropylcellulose | — | 0.75 | — | 0.75 | — | 0.75 | 0.75 |
| Propylene Glycol | — | — | 25 | — | 25 | 25 | 25 |
| Water | 4 | 4 | 4 | 4 | 4 | 4 | 4 |
| Ethanol | q.s. | q.s. | q.s. | q.s. | q.s. | q.s. | q.s. |
| Viscosity (2.5/sec) | 51 | 663 | 26 | 8,440 | 484 | 1,370 | 15,400 |
| cps | | | | | | | |

The formulations were prepared as follows: ETD 2020 when present in the formulation was dispersed in methyl salicylate with stirring (Mixture A). The menthol was dissolved in the ethanol-water mixture, and then hydroxypropylcellulose, if present in the formulation, was then added to this mixture and allowed to swell with stirring (Mixture B). Methyl salicylate or Mixture A was then added to Mixture B, after which propylene glycol, if present in the formulation, was added and mixed until homogeneous.

Comparing Example 2-D with Examples 2-A and 2-B clearly shows the synergy of hydroxypropylcellulose and the acrylic acid-based polymer ETD. 2020. Examples 2-E, 2-C, and 2-A show that propylene glycol does not exhibit synergism with the acrylic acid-based polymer. Examples 2-F, 2-B, and 2-C demonstrate that propylene glycol when combined with hydroxypropylcellulose show very mild synergism. However, comparing Example 2-G with Example 2-D shows that propylene glycol is an effective tertiary gelling agent to the already synergistic hydroxypropylcellulose-acrylic acid-based polymer combination.

This example shows that a topical alcoholic gel containing methyl salicylate and menthol can be prepared using hydroxypropylcellulose and a non-neutralized acrylic acid-based polymer as gelling system, with diol as optional tertiary synergistic gelling agent. Methyl salicylate is an analgesic while menthol is both a counter-irritant and an antipruritic agent.

### EXAMPLE 3

| | 3-A | 3-B | 3-C | 3-D | 3-E | 3-F | 3-G | 3-H |
|---|---|---|---|---|---|---|---|---|
| Ingredients | % w/w | % w/w | % w/w | % w/w | % w/w | % w/w | % w/w | % w/w |
| Methyl salicylate | 10 | 10 | 10 | 10 | 10 | 10 | 10 | 10 |
| Menthol | 7 | 7 | 7 | 7 | 7 | 7 | 7 | 7 |
| ETD 2020 | 1 | 1 | 1 | — | — | 1 | 1 | 1 |
| Hydroxypropyl | — | — | — | 0.75 | 0.75 | 0.75 | 0.75 | 0.75 |
| Cellulose | | | | | | | | |
| Butylene Glycol | — | 25 | — | 25 | — | — | 25 | — |
| Glycerin | — | — | 25 | — | 25 | — | — | 25 |
| Water | 4 | 4 | 4 | 4 | 4 | 4 | 4 | 4 |
| Ethanol | q.s. | q.s. | q.s. | q.s. | q.s. | q.s. | q.s. | q.s. |
| Viscosity (2.5/sec) | 51 | 204 | 102 | 791 | 1,140 | 8,440 | 17,700 | 13,400 |
| cps | | | | | | | | |

The formulations were prepared as in Example 2 but with glycerin or butylene glycol replacing propylene glycol.

Comparing Examples 3-B and 3-A shows no significant synergism between butylene glycol and the acrylic acid-based polymer ETD 2020. Likewise, comparing Examples 3-C and 3-A shows no significant synergism between glycerin and the acrylic acid-based polymer ETD 2020. Examples 3-D and 3-E demonstrate no strong binary synergism of hydroxypropylcellulose with butylene glycol or glycerin. Finally, comparing Examples 3-G and 3-H with 3-F clearly shows that butylene glycol or glycerin is an effective tertiary gelling agent to the already synergistic hydroxypropylcellulose-acrylic acid-based polymer combination.

The results of Examples 2 and 3 indicate that diols and triols, and naturally by extension mixtures thereof, can be used as an effective tertiary gelling agent to the already synergistic hydroxypropylcellulose-acrylic acid-based polymer combination.

### EXAMPLE 4

| | | 4-B | 4-C | | |
|---|---|---|---|---|---|
| | 4-A | % | % | 4-D | 4-E |
| Ingredients | % w/w | w/w | w/w | % w/w | % w/w |
| ETD 2020 | — | 1 | — | 1 | — |
| Ultrez 10 | — | | 1 | | 1 |
| Hydroxypropylcellulose | 0.75 | — | — | 0.75 | 0.75 |
| Water | 5 | 5 | 5 | 5 | 5 |
| Ethanol | q.s. | q.s. | q.s. | q.s. | q.s. |
| Viscosity (cps @ | 510 | 51 | 26 | 4,740 | 3,360 |
| 2.5/sec) | | | | | |

The formulations were prepared as follows: the polymer(s) was added to the ethanol-water mixture while stirring and then mixed until homogeneous.

Ultrez 10 is a homopolymer of acrylic acid, while ETD 2020 is a copolymer of acrylic acid and methyl methacrylate. The results above show that both homopolymer and copolymer of acrylic acids are synergistic with hydroxypropylcellulose indicating that the acrylic acid portion of the polymer is the one contributing to the synergism.

### EXAMPLE 5

| | | | | | 5-E |
|---|---|---|---|---|---|
| | 5-A | 5-B | 5-C | 5-D | % |
| Ingredients | % w/w | % w/w | % w/w | % w/w | w/w |
| Methyl salicylate | 10 | 10 | 10 | 10 | 10 |
| Menthol | 7 | 7 | 7 | 7 | 7 |
| ETD 2020 | 1 | 1 | 1 | 1 | 1 |
| Hydroxypropyl- | 0.75 | — | — | — | — |
| cellulose | | | | | |
| Hydroxypropyl- | | 0.75 | — | — | — |
| methylcellulose | | | | | |
| Carboxymethylcellulose | | | 0.75 | — | — |
| Hydroxyethylcellulose | | | | 0.75 | — |
| Polyvinylpyrrolidone | | | | — | 0.75 |
| Propylene Glycol | 25 | 25 | 25 | 25 | 25 |
| 95% Ethanol | q.s. | q.s. | q.s. | q.s. | q.s. |
| Viscosity (cps @ | 15,400 | 433 | 331 | 25 | 26 |
| 2.5/sec) | | | | | |

The formulations were prepared as follows: ETD 2020 was dispersed in methyl salicylate with stirring (Mixture A). The menthol was dissolved in 95% ethanol, and the second polymer was then added to this mixture and allowed to swell with stirring (Mixture B). Mixture A was then added to Mixture B, after which propylene glycol was added and mixed until homogeneous.

The results show that hydroxypropylmethylcellulose, hydroxyethylcellulose, carboxymethylcellulose, and polyvinylpyrrolidone do not exhibit synergism with the acrylic acid-based polymer. Only hydroxypropylcellulose exhibits synergism with an acrylic acid-based polymer in alcoholic gels.

### EXAMPLE 6

| | 6-A | 6-B | 6-C | 6-D | 6-E | 6-F |
|---|---|---|---|---|---|---|
| Ingredients | % w/w | % w/w | % w/w | % w/w | % w/w | % w/w |
| ETD 2020 | 1 | 1 | 1 | 1 | 1 | 1 |
| Hydroxypropylcellulose | — | — | — | 0.75 | 0.75 | 0.75 |
| Tetrahydroxypropylethylenediamine | 0.75 | 1.5 | 2.3* | 0.75 | 1.5 | 0.75 |
| Propylene Glycol | — | — | — | — | — | 10 |
| Water | 5 | 5 | 5 | 5 | 5 | 5 |
| Ethanol | q.s. | q.s. | q.s. | q.s. | q.s. | q.s. |
| Viscosity (cps @ 2.5/sec) | 25 | 7,420 | 11,600 | 12,100 | 30,900 | 25,600 |

*Complete neutralization of ETD 2020

The formulations Were prepared as follows: hydroxypropylcellulose when present in the formulation and ETD 2020 were added to the ethanol-water mixture with stirring. When the polymer(s) was fully hydrated, propylene glycol, if present in the formulation, was added and mixing continued until the mixture was homogeneous. Tetrahydroxypropylethylenediamine was then added and the final composition mixed to homogeneity.

Example 6-C is a typical example of how alcoholic gels are formed in prior art, i.e. by fully neutralizing a high level of ETD 2020.

The results indicate that hydroxypropylcellulose can be used synergistically with partially neutralized ETD 2020 and still provides higher viscosity than equivalent level of fully neutralized ETD 2020 (compare 6-D and 6-E vs. 6-C), allowing one to formulate alcoholic gels with lower levels of base. Moreover, addition of a diol, such as propylene glycol, significantly increases the viscosity of the hydroxypropylcellulose and partially neutralized ETD 2020 combination (compare 6-F vs. 6-D). The gelling system of the present invention allows one to use lower levels of base but still achieve higher viscosity in alcoholic gels than what is possible with the fully neutralized acrylic acid-based polymer of prior art (compare 6-D, 6-E and 6-F with 6-C).

### EXAMPLE 7

| | 7-A | 7-B | 7-C | 7-D | 7-E |
|---|---|---|---|---|---|
| Ingredients | % w/w | % w/w | % w/w | % w/w | % w/w |
| Methyl salicylate | 10 | 10 | 10 | 10 | 10 |
| Menthol | 7 | 7 | 7 | 7 | 7 |
| ETD 2020 | 1 | 1 | 0.3 | 0.5 | 0.5 |
| Hydroxypropylcellulose | — | 0.75 | 0.75 | 0.75 | 0.75 |
| Tetrahydroxypropylethylenediamine | 2.3* | 0.75 | 0.7* | 1.15* | 1.15* |
| Propylene Glycol | — | 25 | 25 | — | 25 |
| Water | 4 | 4 | 4 | 4 | 4 |
| Ethanol | q.s. | q.s. | q.s. | q.s. | q.s. |
| Viscosity (cps @ 2.5/sec) | 13,200 | 29,900 | 12,900 | 23,700 | 31,300 |

*Complete neutralization of ETD 2020

The formulations were prepared as follows: ETD 2020 was dispersed in methyl salicylate with stirring (Mixture A). The menthol was dissolved in the ethanol-water mixture, and then hydroxypropylcellulose, if present in the formulation, was then added to this mixture and allowed to swell with stirring (Mixture B). Mixture A was then added to Mixture B, after which propylene glycol, if present in the formulation, was added and mixed until homogeneous. Tetrahydroxypropylethylenediamine was then added and the final composition mixed to homogeneity.

Example 7-A is a typical example of how alcoholic gels are formed in prior art, i.e. by fully neutralizing a high level of ETD 2020. Example 7-B of this instant invention, which uses hydroxypropylcellulose, equivalent level of ETD 2020 as Example 7-A but only partially neutralized, and propylene glycol as tertiary gelling agent, leads to significantly higher viscosity than Example 7-A, while using a significantly lower level of base.

Example 7-C is another embodiment of the present invention, where fully neutralized ETD 2020 is used with hydroxypropylcellulose and propylene glycol. Note that Example 7-C contains significantly reduced amount of ETD 2020 and base compared to Example 7-A, but the viscosities are about the same.

Example 7-D is another embodiment of the present invention, where fully neutralized ETD 2020 is used with hydroxypropylcellulose without the optional diol and/or triol as tertiary gelling agent. Note that Example 7-D contains significantly reduced amount of ETD 2020 and base compared to Example 7-A, but the viscosity of Example 7-D is almost twice as high as Example 7-A. Addition of the tertiary gelling agent propylene glycol to Example 7-D results in even higher viscosity (compare 7-E with 7-D).

### EXAMPLE 8

| | Example 8-A | Example 8-B |
|---|---|---|
| Ingredients | % w/w | % w/w |
| Ibuprofen | 2.5 | 2.5 |
| ETD 2020 | 1.5 | 1.5 |
| Hydroxypropylcellulose | 1.1 | 1.1 |
| Propylene Glycol | 25 | 25 |
| Tetrahydroxypropylethylenediamine | — | 1.1 |
| 95% Ethanol | q.s. | q.s. |
| Viscosity (cps @ 2.5/sec) | 20,400 | 42,200 |

The formulations were prepared by first dissolving ibuprofen in 95% ethanol. ETD 2020 and hydroxypropylcellulose were then dispersed in this mixture with stirring, followed by the addition of propylene glycol, and mixing to homogeneity. Tetrahydroxypropylethylenediamine when present in the formulation was then added and the final composition mixed to homogeneity.

Ibuprofen is a non-steroidal anti-inflammatory drug. This example shows that topical ibuprofen alcoholic gels can be prepared by using the gelling system of the present invention. Example 8-A uses non-neutralized ETD 2020 while Example 8-B uses partially neutralized (32% neutralization) ETD 2020.

### EXAMPLE 9

| Ingredients | % w/w |
|---|---|
| Lidocaine | 5 |
| ETD 2020 | 1 |
| Hydroxypropylcellulose | 0.75 |
| 95% Ethanol | q.s. |
| Viscosity (2.5/sec) | >10,000 cps |

The formulation was prepared by first dissolving lidocaine in 95% ethanol. ETD 2020 and hydroxypropylcellulose were then dispersed in this mixture with stirring until homogeneous.

This example shows that a topical lidocaine alcoholic gel can be prepared by using the gelling system of the present invention. Lidocaine is a topical anesthetic.

### EXAMPLE 10

| Ingredients | % w/w |
|---|---|
| Prilocaine | 5 |
| ETD 2020 | 1 |
| Hydroxypropylcellulose | 0.75 |
| Glycerin | 10 |
| Propylene glycol | 10 |
| 95% Ethanol | q.s. |
| Viscosity (2.5/sec) | >15,000 cps |

The formulation was prepared by first dissolving prilocaine in 95% ethanol. ETD 2020 and hydroxypropylcellulose were then dispersed in this mixture with stirring, followed by the addition of glycerin and propylene glycol, and mixing to homogeneity.

This example shows that a topical prilocaine alcoholic gel can be prepared by using the gelling system of the present invention. Prilocaine is a topical anesthetic.

### EXAMPLE 11

| | Example 11-A | Example 11-B |
|---|---|---|
| Ingredients | % w/w | % w/w |
| Betamethasone valerate | 0.1 | 0.1 |
| ETD 2020 | 1 | 1 |
| Hydroxypropylcellulose | 0.75 | 0.75 |
| Glycerin | 25 | 25 |
| Tetrahydroxypropylethylenediamine | — | 0.8 |
| 95% Ethanol | q.s. | q.s. |
| Viscosity (cps @ 2.5/sec) | 11,900 | 37,500 |

The formulations were prepared by first dissolving betamethasone valerate in 95% ethanol. ETD 2020 and hydroxypropylcellulose were then dispersed in this mixture with stirring, followed by the addition of glycerin, and mixing to homogeneity. Tetrahydroxypropylethylenediamine when present in the formulation was then added and the final composition mixed to homogeneity.

Betamethasone valerate is an anti-inflammatory corticosteroid. This example shows that topical betamethasone valerate alcoholic gels can be prepared by using the gelling system of the present invention. Example 11-A uses non-neutralized ETD 2020 while Example 11-B uses partially neutralized (35% neutralization) ETD 2020.

### EXAMPLE 12

| | Example 12-A | Example 12-B |
|---|---|---|
| Ingredients | % w/w | % w/w |
| Capsaicin | 0.03 | 0.03 |
| ETD 2020 | 1 | 1 |
| Hydroxypropylcellulose | 0.75 | 0.75 |
| Butylene glycol | 15 | 15 |
| Tetrahydroxypropylethylenediamine | — | 0.7 |
| 95% Ethanol | q.s. | q.s. |
| Viscosity (cps @ 2.5/sec) | 7,040 | 29,500 |

The formulations were prepared by first dissolving capsaicin in 95% ethanol. ETD 2020 and hydroxypropylcellulose were then dispersed in this mixture with stirring, followed by the addition of butylene glycol, and mixing to homogeneity. Tetrahydroxypropylethylenediamine when present in the formulation was then added and the final composition mixed to homogeneity.

Capsaicin is an analgesic useful for temporary relief of muscle and joint pains associated with arthritis. This example shows that topical capsaicin alcoholic gels can be prepared by using the gelling system of the present invention. Example 12-A uses non-neutralized ETD 2020 while Example 12-B uses partially neutralized (30% neutralization) ETD 2020.

While the present invention has been described in terms of its specific embodiments, certain modifications and equivalents will be apparent to those skilled in the art and are intended to be included within the scope of the present invention.

We claim:

1. An alcoholic gel for topical application, which comprises:
(a). an effective amount of a pharmaceutical active ingredient other than piroxicam;
(b). greater than 40% w/w of lower alkanol having from two to three carbon atoms;
(c). from about 0.2% w/w to about 2% w/w of hydroxypropylcellulose;
(d). from about 0.2% w/w to about 2% w/w of an acrylic acid-based polymer; and
(e). less than 20% w/w of water.

2. The composition according to claim 1, wherein the pharmaceutical active ingredient is present from about 0.02% w/w to about 30% w/w.

3. The composition according to claim 2, wherein the pharmaceutical active ingredient is selected from the group consisting of analgesic, counter-irritants, anti-inflammatory agents, antipruritics, anesthetics, antifungals, antiviral agents, antiobiotics, and mixtures thereof.

4. The composition according to claim 3, wherein said pharmaceutical active ingredient is selected from the group consisting of methyl salicylate, menthol, camphor, capsaicin and its derivatives, diphenhydramine and its salts, ibuprofen, ketoprofen, diclofenac, benzocaine, lidocaine, lidocaine hydrochloride, prilocaine, prilocaine hydrochloride, hydrocortisone, hydrocortisone aceponate, hydrocortisone acetate, hydrocortisone butyrate, hydrocortisone valerate, betamethasone dipropionate, betamethasone valerate, clobetasol propionate, flucinolone acetonide, triamcinolone acetonide, halobetasol propionate, and mixtures thereof.

5. The composition according to claim 1, wherein said lower alkanol is ethanol.

6. The composition according to claim 1, wherein the hydroxypropylcellulose is present from about 0.3% w/w to about 1.3% w/w.

7. The composition according to claims 1, wherein the acrylic acid-based polymer is present from about 0.3% w/w to about 1.3% w/w.

8. The composition according to claim 1, wherein the water is less than 15% w/w.

9. The composition according to claim 8, wherein the water is less than 10% w/w.

10. The composition according to claim 1, which further comprises an amine base at a concentration less than 2% w/w.

11. The composition according to claim 10, wherein the amine base is present at a concentration less than 1% w/w.

12. The composition according to claim 10, wherein the amine base is selected from tetrahydroxypropylethylenediamine, diisopropanolamine, triisopropanolamine, and mixtures thereof.

13. The composition according to claim 1, which further comprises from about 5% w/w to about 35% w/w of a diol, triol, or mixtures thereof.

14. The composition according to claim 13, wherein the diol and triol are selected from the group consisting of propylene glycol, butylene glycol, hexylene glycol, glycerin, and mixtures thereof.

15. An alcoholic gel for topical application, which comprises:
(a). 0.02-30% w/w of a pharmaceutical active ingredient other than piroxicam;
(b). greater than 40% w/w of ethanol;
(c). 0.3-1.3% w/w of hydroxypropylcellulose;
(d). 0.3-1.3% w/w of an acrylic acid-based polymer;
(e). an amount of amine base less than or equal to about 60% of what is required to neutralize the acrylic acid-based polymer; and
(f). 0-15% w/w of water.

16. The composition according to claim 15, which further comprises from about 5% w/w to about 35% w/w of a diol, triol, or mixtures thereof.

17. An alcoholic gel for topical application, which comprises:
(a). 0.02-30% w/w of a pharmaceutical active ingredient other than piroxicam;
(b). greater than 40% w/w of ethanol;
(c). 0.3-1% w/w of hydroxypropylcellulose;
(d). 0.3-0.7% w/w of an acrylic acid-based polymer;
(e). an amount of amine base to fully neutralize the acrylic acid-based polymer; and
(f). 0-15% w/w of water.

18. The composition according to claim 17, which further comprises from about 5% w/w to about 35% w/w of a diol, triol, or mixtures thereof.

19. An alcoholic gel for topical application, which comprises:
(a). 10-20% w/w methyl salicylate;
(b). 2-10% w/w menthol;
(c). 40-80% w/w ethanol;
(c). 10-30% w/w of a diol and/or triol;
(d). 0.3-1.3% w/w hydroxypropylcellulose;
(e). 0.3-1.3% w/w acrylic acid-based polymer; and
(f). 0-15% w/w of water.

20. The composition according to claim 19, which further comprises less than about 1.5% w/w of an amine base selected from tetrahydroxypropylethylenediamine, diisopropanolamine, triisopropanolamine, and mixtures thereof.
